(19) United States
(12) Patent Application Publication
(10) Pub. No.: US 2003/0014071 A1
(43) Pub. Date: Jan. 16, 2003

(54) Inflation device and methods of use

(76) Inventors: Timothy C. Reynolds, Sunnyvale, CA (US); Daniel H. Shumer, San Jose, CA (US); Travis R. Yribarren, San Mateo, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/150,824

(22) Filed: May 20, 2002

Related U.S. Application Data
(60) Provisional application No. 60/305,550, filed on Jul. 14, 2001.

(51) Int. Cl. A61M 29/00

(52) U.S. Cl. 606/192

(57) ABSTRACT

A device for inflating balloons disposed upon medical devices, wherein the balloons require accurate volume inflation. The device including a main body, a seal lever, an inflation knob coupled to a fluid chamber. The device further includes a first seal knob disposed within the main body, wherein the seal knob is operatively coupled to the seal lever. A second seal knob disposed within a shuttle assembly coupled to the main body, wherein the second seal knob is operatively coupled to the seal lever; and a shuttle assembly coupled to a locking knob, wherein the shuttle assembly opens an closes a valve assembly disposed upon a medical device inserted into a valve chamber of the main body.

### PRIORITY CLAIM

The present invention claims priority to U.S. Provisional Patent Application Serial No. 60/305,550 filed Jul. 14, 2002, wherein the above-referenced application is hereby incorporated by reference in its entirety.

### FIELD OF THE INVENTION

The present invention relates to inflation devices, more particularly the present invention relates to inflation devices utilized to inflate an inflatable medical device, for example, the inflation device may be utilized to inflate a balloon disposed upon a catheter, guidewire, cannula, or similar medical devices.

### DESCRIPTION OF THE PRIOR ART

Medical devices having inflatable balloons contained thereon are commonly utilized for many procedures. For example, a catheter having an inflatable balloon disposed thereupon may be utilized to occlude a vessel, expand a vessel, or deploy a medical device such as a stent. During these procedures, typically a guidewire is advanced to an area where it is desired to deploy an inflatable balloon. A catheter or similar device having a balloon mounted thereupon is then advanced over the guidewire until the balloon is positioned as desired. The balloon is then inflated by filling the chamber of the balloon with an inflation fluid, typically saline or contrast solution. Typically, the balloon is inflated to a known pressure, wherein an undetermined volume of fluid is used to obtain the desired diameter and pressure.

These balloons are typically constructed of a compliant material such as C-Flex, urethane or polyvinyl chloride, or similar materials where the durromoeter and expansion and contraction forces may be controlled. The outer surfaces of the balloon must be smooth and contain no rough edges or areas, which may abrade the vessel wall or cause trauma to the vessel. Additionally, it is desirable to provide a balloon, which has a low profile when no inflation fluid has been introduced into the chamber of the balloon. The benefit of having a to low profile balloon is to provide better tractability or maneuverability of the medical device to which the balloon is affixed. A potential side effect of have a low profile balloon is that due to the materials durrometer, a greater amount of fluid pressure is necessary to inflate the balloon. Typically balloons are inflated using a syringe filled within inflation fluid wherein the syringe is in fluid communication with the chamber of the balloon.

While a syringe coupled to the chamber of the balloon is an effective method of inflating the balloon there are some dangers associated with this method. Particularly, it is difficult to control the diameter of the inflated balloon through the use of a syringe because a small change in volume and pressure within the syringe may translate to a larger force within the chamber of the balloon. For example, if it is desirable to inflate the balloon to 5 mm, the user must inject a given amount of fluid into the chamber of the balloon and retain that amount by closing a valve or holding the syringe plunger in a fixed position. Though, in the process of closing the valve, the user may apply a force to the syringe therein causing the balloon to inflate to a diameter greater than what is desired, or alternatively to a diameter smaller than desired wherein the vessel is not occluded properly. Still further, even though the manufacturer of the medical device having the balloon disposed thereupon may supply a syringe the supplied syringe may not be utilized to inflate the balloon. Therefore, it is possible that a syringe having a different diameter barrel or markings on the barrel which are slightly different than those on the supplied syringe which may lead to over/under inflation and/or damage to the vessel.

Further still, with recent advancements it has become possible to form a guidewire having an inflatable balloon and a valve that may be selectively opened and closed for inflation/deflation of the balloon. While this device eliminates the need for a separate occlusion catheter it presents other problems in that a syringe can no longer be utilized to inflate/deflate the balloon without the use of an adapter. Though, adapters are presently available they have many shortcomings, for example they require multiple steps in order to prepare and use them which introduces many points for errors to be made in preparing the system. Additionally, these adapters further require the user to utilize a separate syringe that must be attached to the adapter to perform inflation/deflation of the balloon.

Therefore, there is a need for an apparatus that will allow a user to accurately inflate/deflate a balloon. Furthermore, there is a need for an apparatus that will allow the balloon to be accurately inflated/deflated repeatedly.

There is also a need for an inflation device that allows the user to inflate the balloon in small increments accurately, such that the overall diameter of the inflated balloon may be carefully controlled.

There is also a need for an inflation device that is easy to operate and does not require multiple pieces to be assembled during a surgical procedure in order to utilize the inflation device.

Still further, there is a need for an inflation device which may be utilized to open a valve assembly of a medical device, accurately inflate a balloon to a desired diameter, and then be removed from the medical device so that the medical device may-be utilized for other procedures, such as being utilized to occlude a vessel with the inflated balloon as well as serve the function of a guidewire.

### SUMMARY OF THE PRESENT INVENTION

In accordance with the present invention there is provided a device for inflating balloons disposed upon medical devices, wherein the balloons require accurate volume inflation. The device including a main body, a locking cover, an inflation knob coupled to a fluid chamber, a first seal knob disposed within the main body, wherein the seal knob is operatively coupled to the seal lever, and a second seal knob disposed within a shuttle assembly coupled to the main body, wherein the second seal knob is operatively coupled to the seal lever. The device further includes a shuttle assembly coupled to a locking knob, wherein the shuttle assembly opens and closes a valve assembly disposed upon a medical device inserted into a valve chamber of the main body.

In accordance with yet another embodiment in accordance with the present invention, there is provided an inflation device, wherein the inflation device is configured to actuate a valve assembly of an inflatable medical device. The inflation device includes a main body having a fluid inlet, a vacuum outlet and a fluid reservoir. The inflation device further includes an inflation chamber, wherein the inflation chamber is configured to receive an inflatable medical device therein, wherein the inflation chamber includes a device configured for actuating a valve assembly disposed on the inflatable medical device.

In accordance with the present invention there is provided a method of inflating an inflatable medical device, the method includes the steps of (a) disposing a medical device having an inflation port within an inflation chamber of an inflation device, (b) creating a vacuum within the inflation chamber and within an inflation lumen of the inflatable medical device, wherein the inflation chamber and inflation lumen are in fluid communication with an inflatable balloon disposed on the inflatable medical device; (c) providing an inflation fluid, wherein the inflation fluid is in fluid communication with the inflation lumen; and (d) displacing a predetermined volume of inflation fluid to inflate the inflatable balloon to a known diameter.

In accordance with the present invention there is provided yet another method for inflating a balloon disposed upon a medical device, the medical device including a low profile valve and a balloon disposed upon a distal end portion. The method including the steps of, providing an inflation device, the inflation device comprising a main body, an inflation chamber, a device for opening an closing the valve chamber, and a device for opening the valve assembly of the medical device. Opening the inflation chamber for insertion of a proximal end portion containing a low profile valve. Closing the inflation chamber therein creating a fluid tight chamber about the valve assembly of the medical device and opening the valve of the medical device. Creating a vacuum within the inflation device and medical device. Providing a source of inflation fluid, wherein the vacuum draws the inflation fluid into the inflation device, valve assembly, inflation chamber and a fluid reservoir. Providing an inflation knob coupled to the fluid reservoir, wherein a rotational force applied to the inflation knob causes fluid to enter the inflation chamber and inflate the balloon, closing the valve assembly of the medical device and releasing the proximal end portion of the medical device from the valve chamber.

### DETAILED DESCRIPTION OF THE DRAWINGS

In the following detailed portion of the present description, the invention will be explained in greater detail with reference to the drawings, wherein:

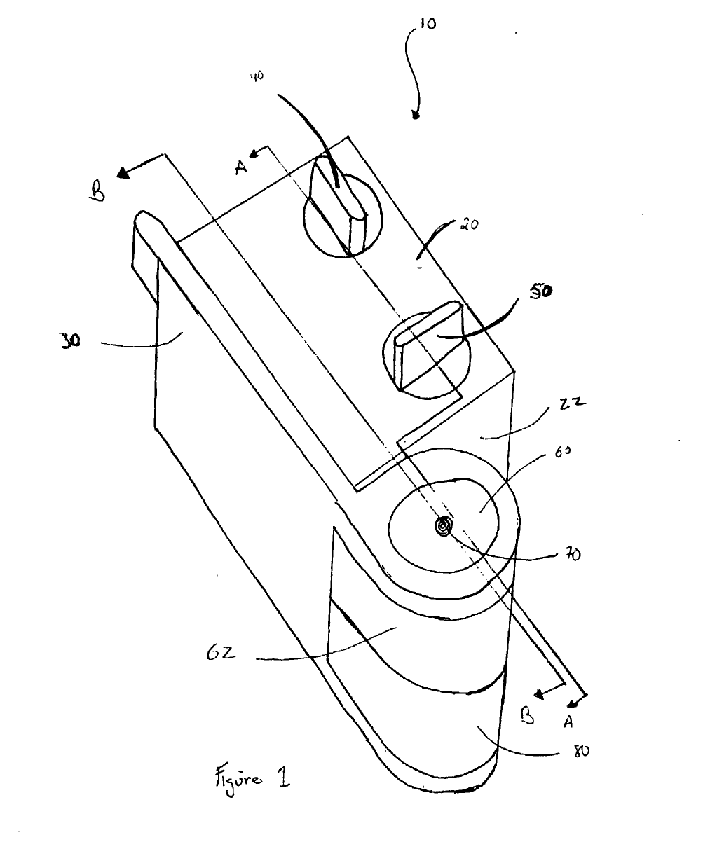

FIG. 1. is an isometric plan view of the inflation device according to the present invention illustrating the seal lever in a closed position;

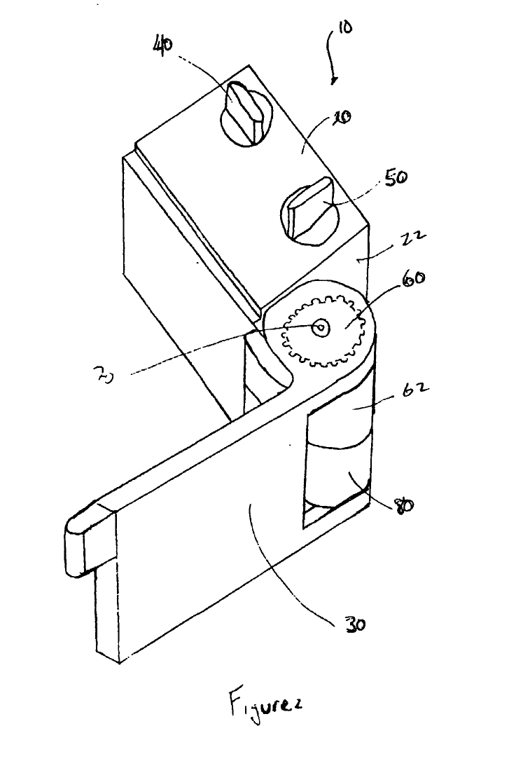

FIG. 2. is an isometric plan view of the inflation device according to the present invention illustrating the seal lever in an open position, wherein the inflation device is capable of receiving a valve assembly of an inflatable medical device;

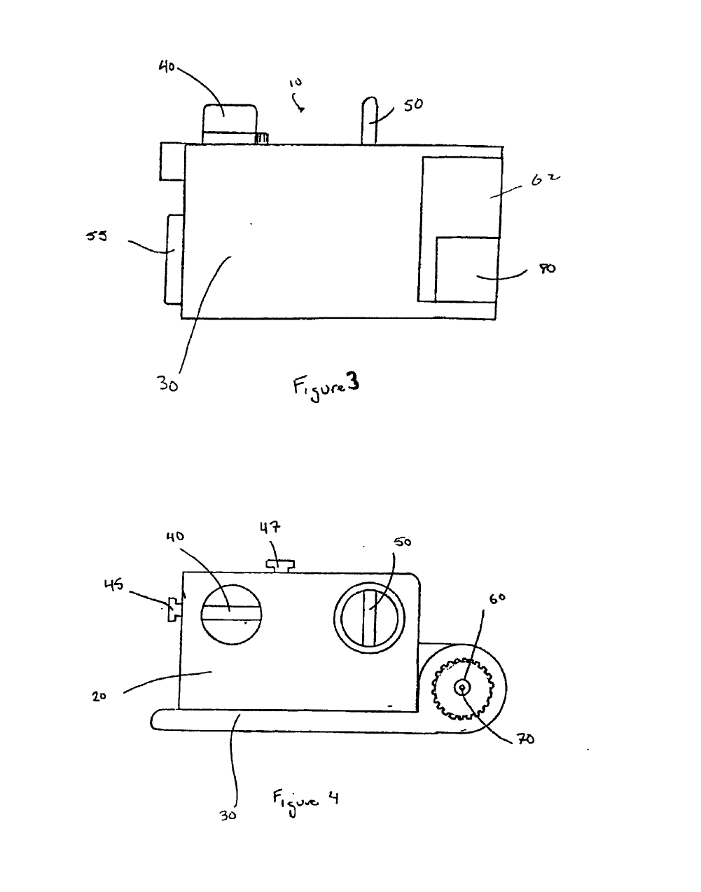

FIG. 3. is a side view of the inflation device according to the present invention;

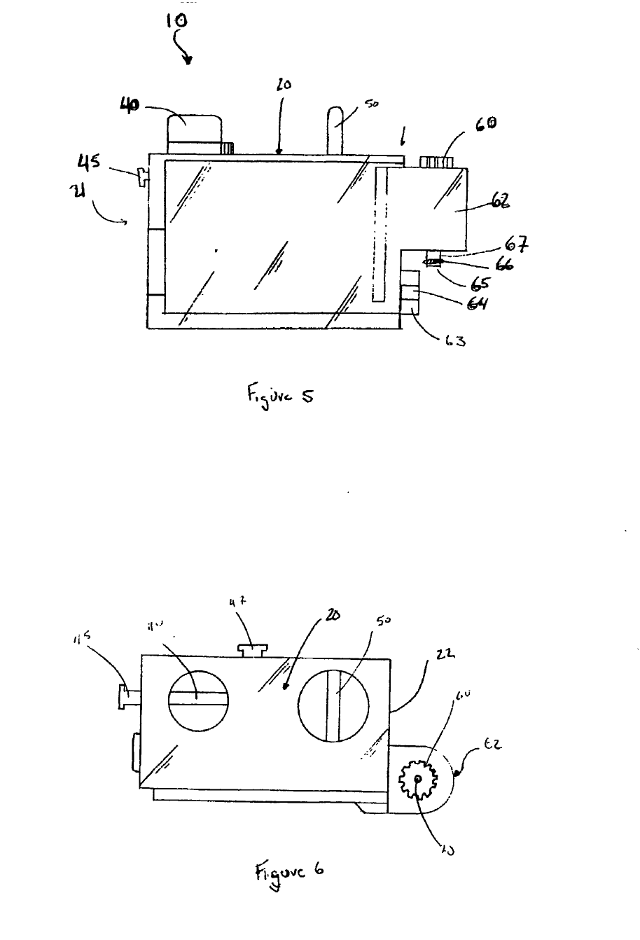

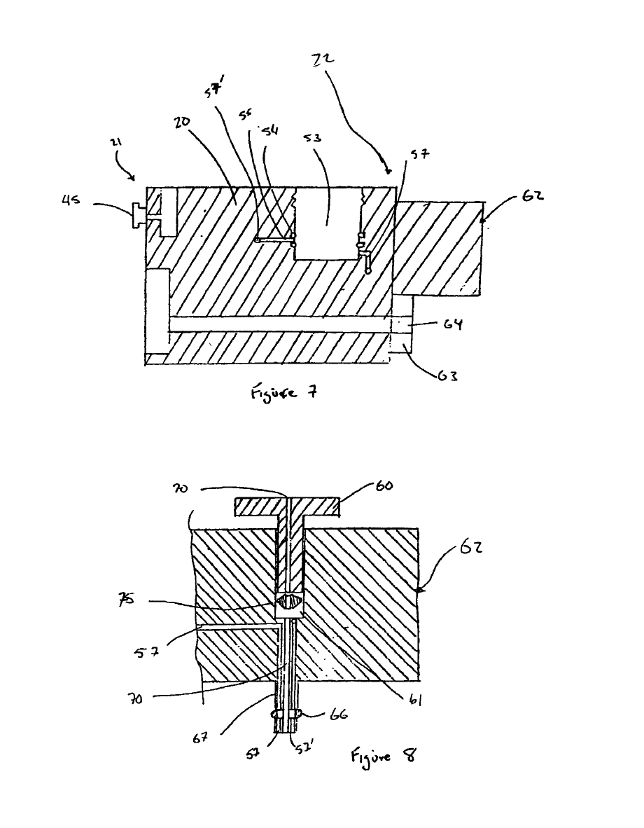

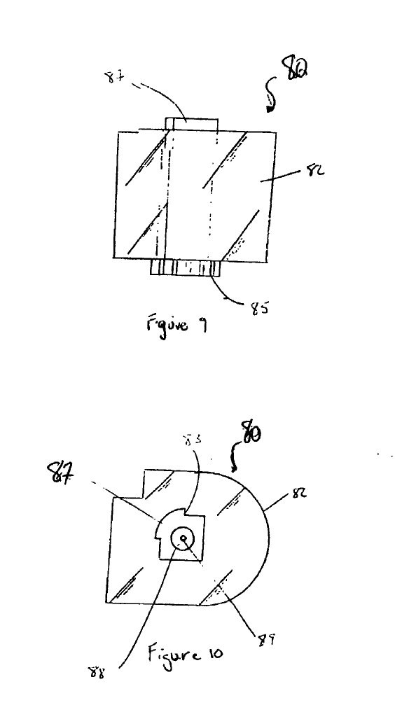

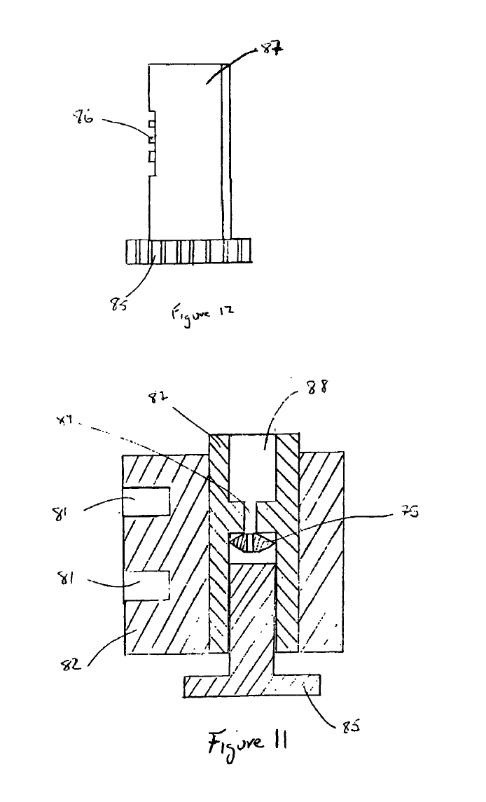

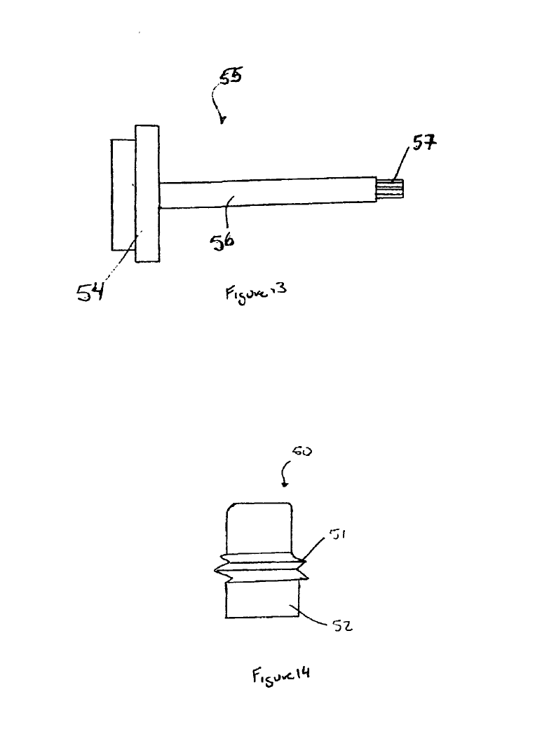

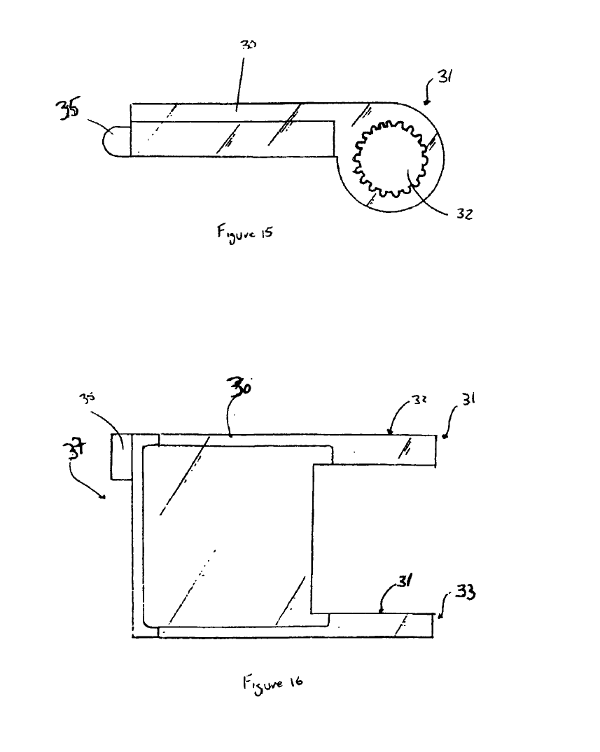

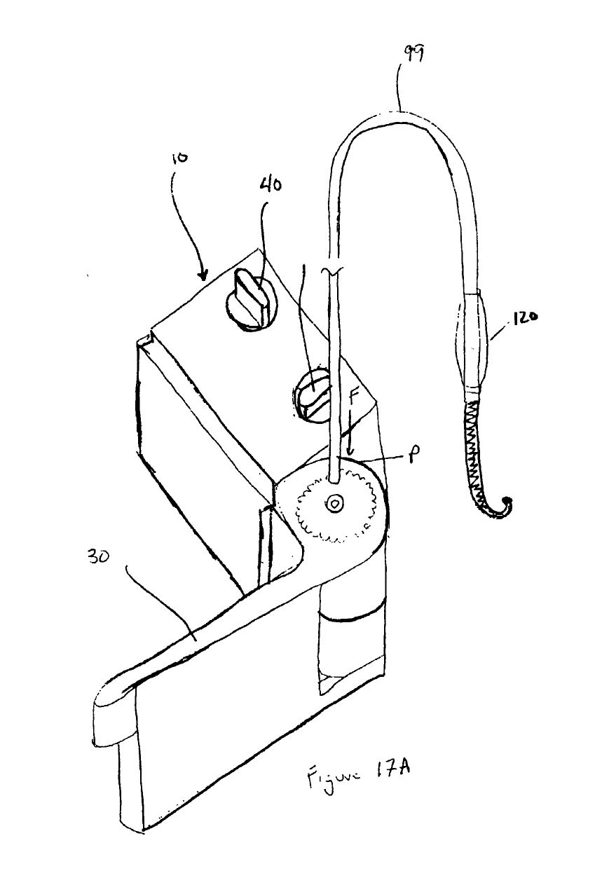

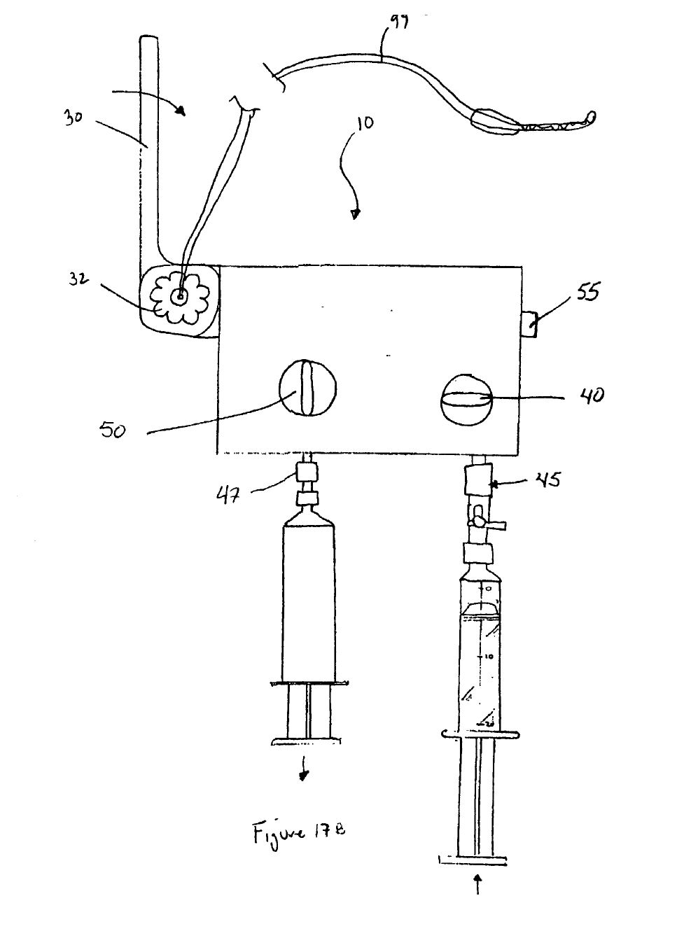

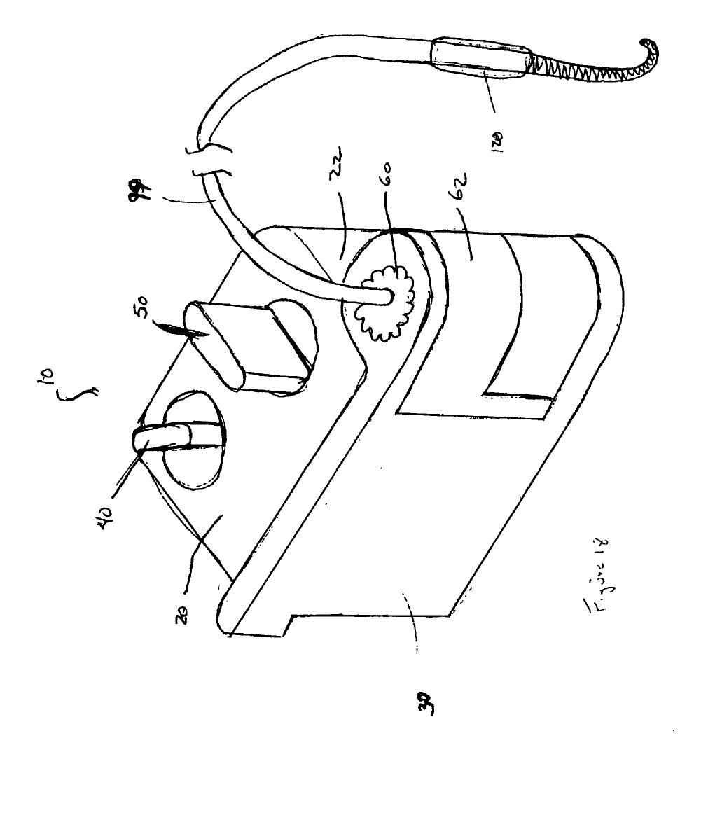

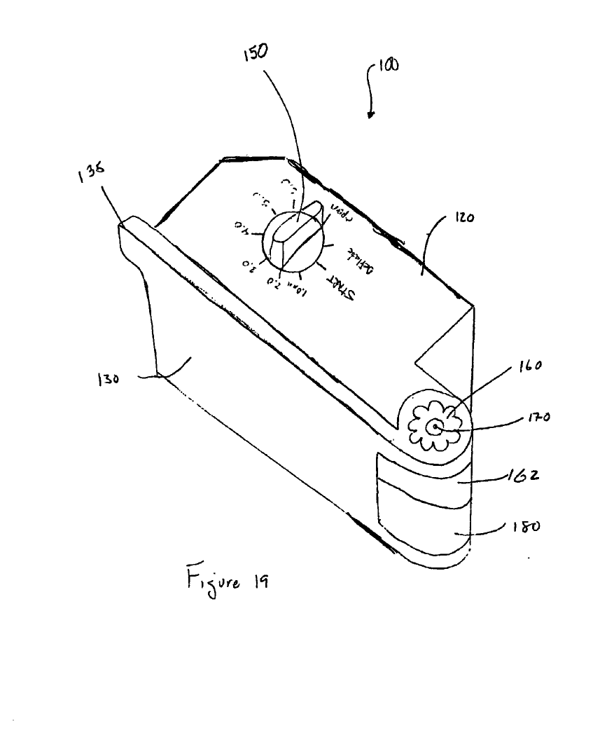

FIG. 4. is a top view of the inflation device according to the present invention;

FIG. 5. is side view of the main body of the inflation device according to the present invention prior to assembly;

FIG. 6. is a top view of the main body illustrating the inflation knob, selector knob, and first seal knob as assembled in the main body;

FIG. 7 Is a cross-sectional side view of the inflation device according to the present invention taken about line A-A of FIG. 1;

FIG. 8 is a partial cross-sectional side view of the projection of the main body illustrating the first seal knob and seal disposed therein, taken about line B-B of FIG. 1;

FIG. 9 is a side view of the shuttle assembly according the present invention;

FIG. 10. is a top view of the shuttle assembly according to the present invention;

FIG. 11. is a cross-sectional side view of the shuttle assembly according to the present invention, wherein there is shown the shuttle body, shuttle, second seal knob, and seal;

FIG. 12. is a side view of the shuttle according to the present invention, wherein a second seal knob is disposed within the proximal end portion of the shuttle;

FIG. 13. is a side view of the locking knob and locking knob rod in accordance with the present invention;

FIG. 14. is a side view of the inflation knob of the inflation device according to the present invention;

FIG. 15. is a bottom view of the seal lever of the inflation device according to the present invention;

FIG. 16. is a side view of the seal lever of the inflation device according to the present invention;

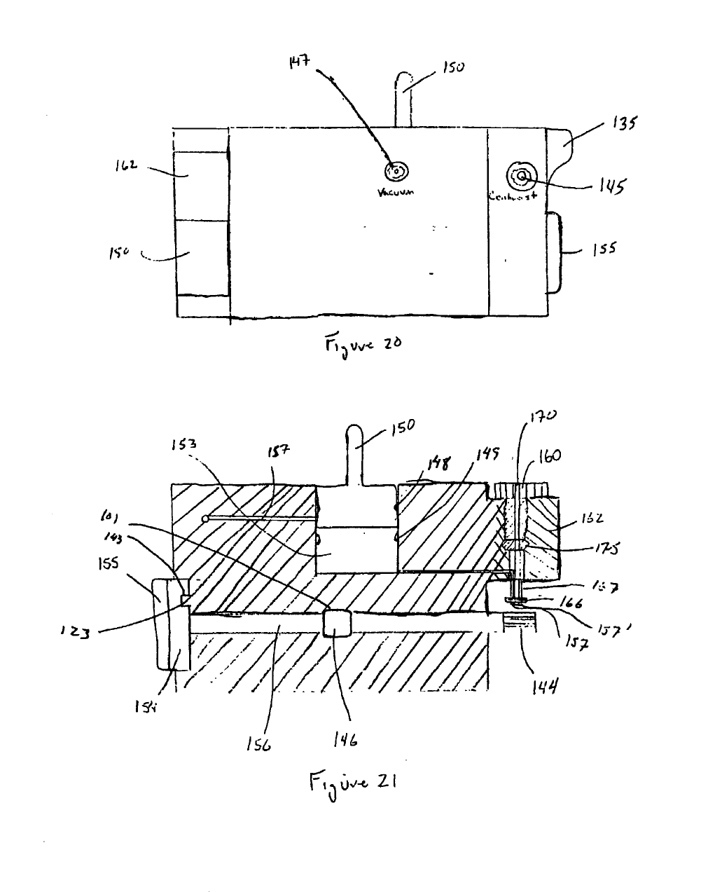

FIG. 17A. is an isometric top view of the inflation device according to the present invention, wherein a medical device having a valve assembly and an inflatable balloon is being inserted within the inflation chamber;

FIG. 17B. is a top view of the inflation device according to the present invention illustrating a vacuum syringe and contrast syringe coupled to the inflation device;

FIG. 18. is an isometric top view of the inflation device according to the present invention, wherein the seal lever and locking knob have been closed therein opening the valve of the medical device for inflation of the balloon;

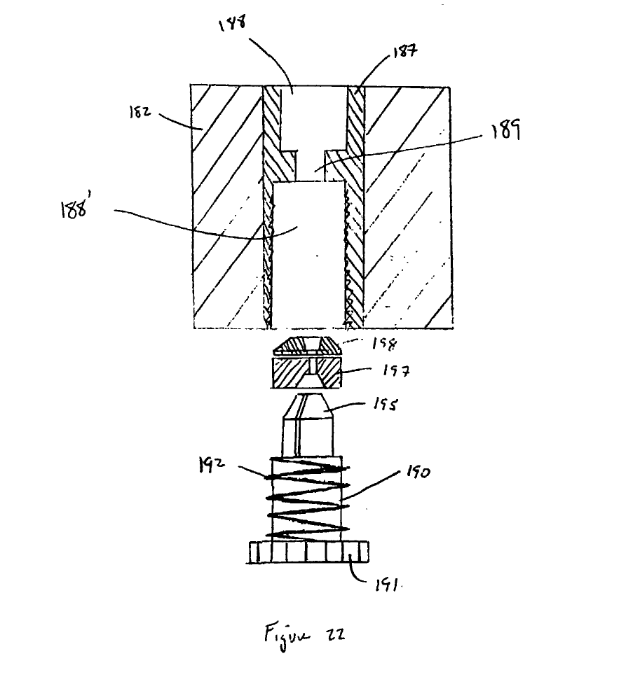

FIG. 19. is an isometric view illustrating an exemplary alternative embodiment of an inflation device in accordance with the present invention;

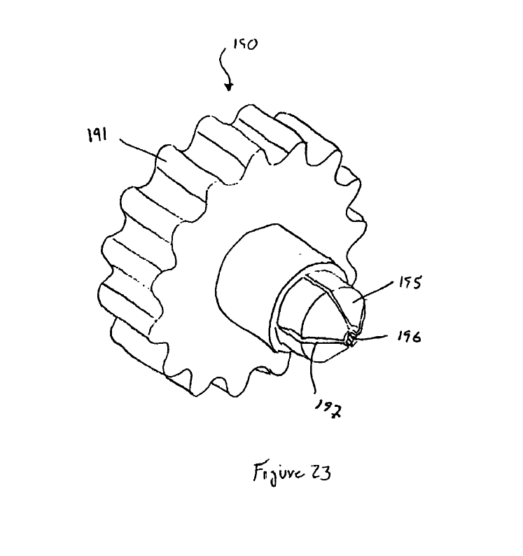

FIG. 20. is a plan side view of the alternative embodiment of the inflation device in accordance with the present invention;

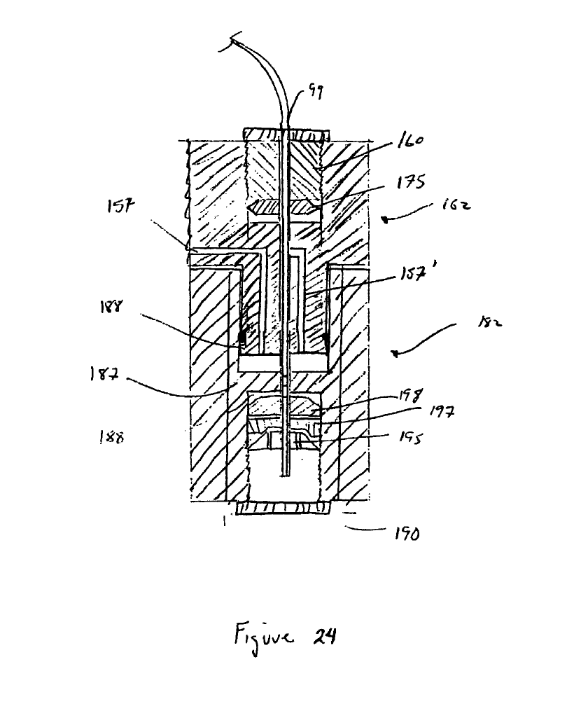

FIG. 21. is a cross-sectional plan view of the main body if the inflation device in accordance with the alternative embodiment of the present invention;

FIG. 22. is a cross-sectional side view of the shuttle assembly in accordance with an alternative embodiment of the present invention;

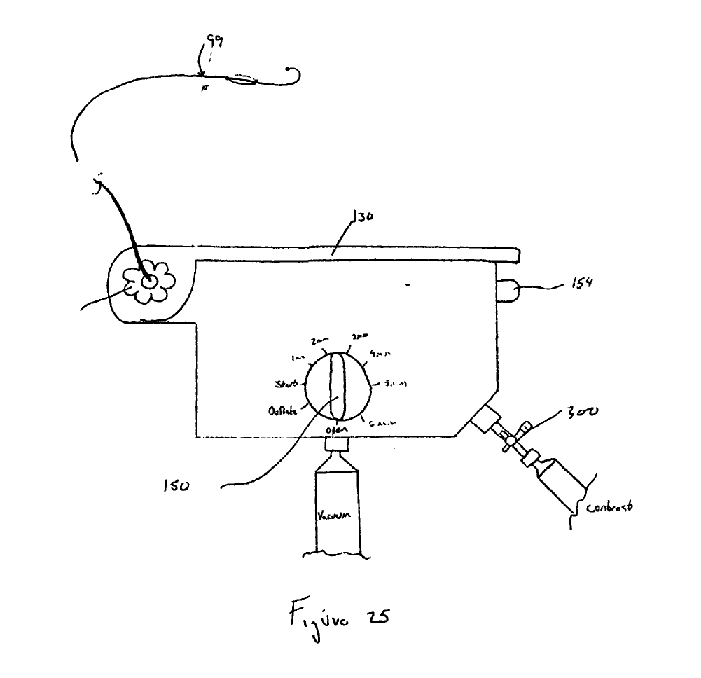

FIG. 23. is an isometric view of the second seal knob in accordance with an alternative embodiment of the present invention;

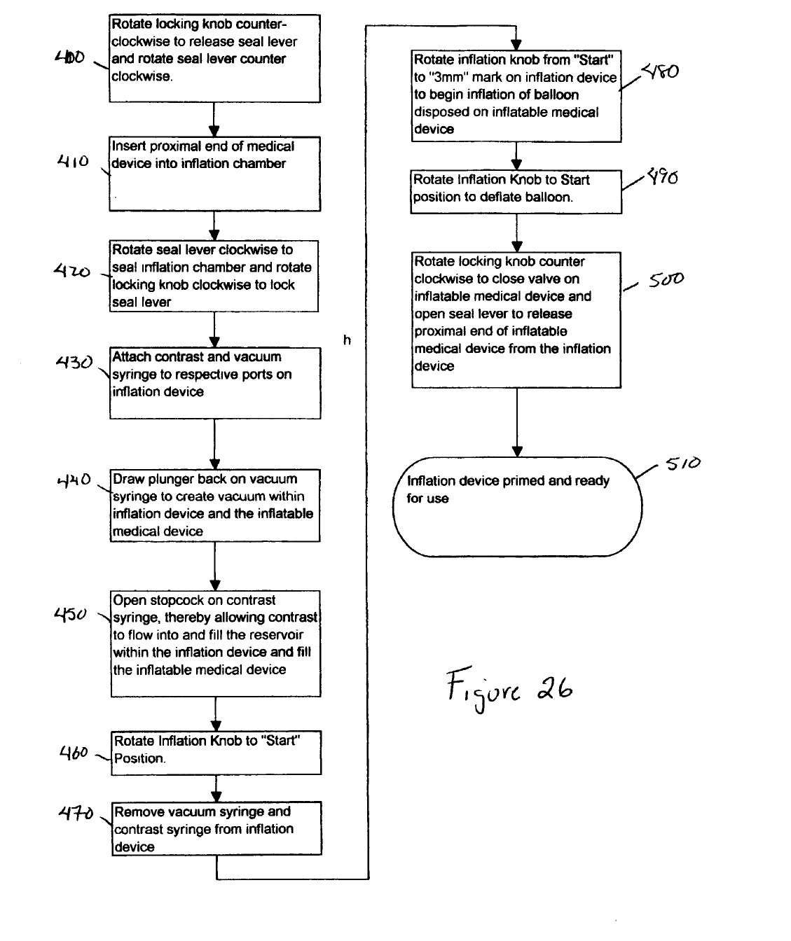

FIG. 24. is a cross-sectional view of the first seal knob, shuttle assembly and second seal knob as assembled in the alternative embodiment of the inflation device;

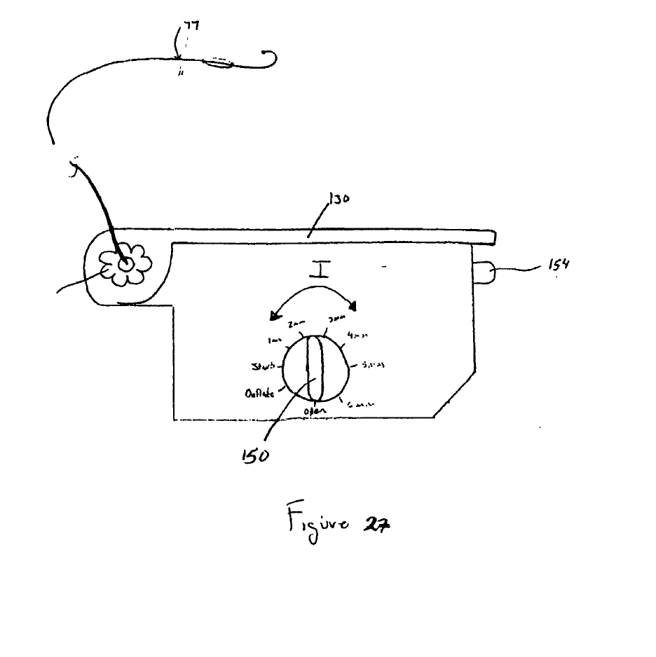

FIG. 25. is a top view illustrating the inflation device prepared for use wherein a proximal end of a medical device to be inflated is disposed within the inflation chamber and an vacuum syringe and contrast syringe are connected to the inflation device;

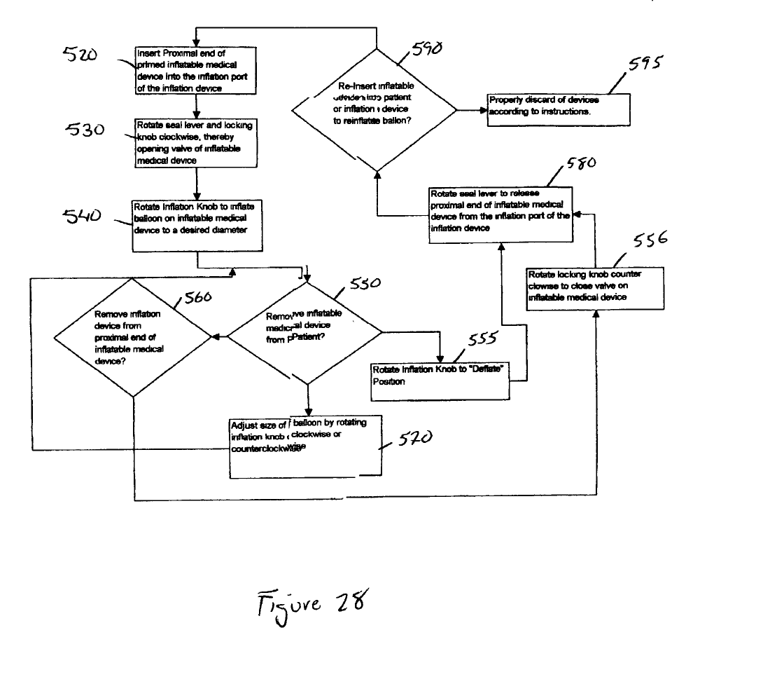

FIG. 26. is a functional flow diagram illustrating a method of use of the present invention;

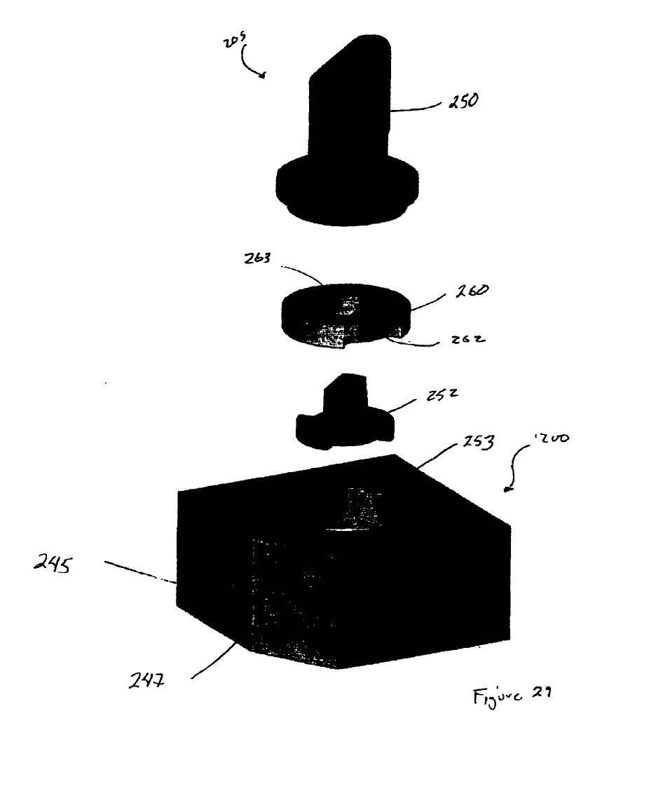

FIG. 27. is a top view illustrating the inflation device prepared for use wherein a proximal end of a medical device to be inflated is disposed within the inflation chamber;

FIG. 28. is a functional flow chart illustrating the method of utilizing the present invention to inflate a balloon on a medical device;

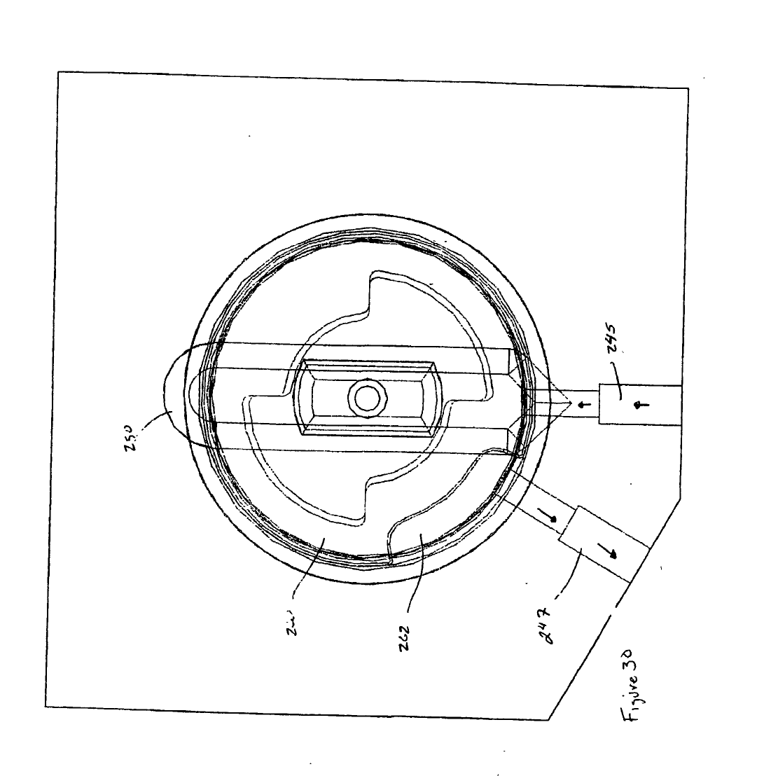

FIG. 29. is an exploded view of an alternative embodiment of the inflation knob incorporating a selector valve in accordance with the present invention;

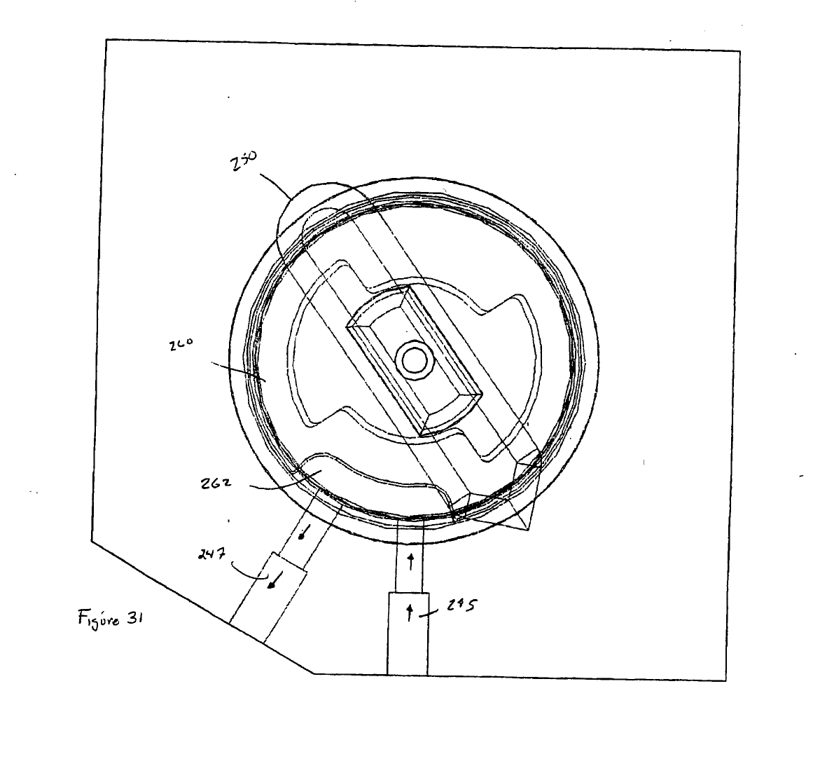

FIG. 30. is a top view of the alternative embodiment of the inflation knob in accordance with the present invention, wherein the selector valve is opened to the vacuum port;

FIG. 31. is a top view of the alternative embodiment of the inflation knob in accordance with the present invention, wherein the selector valve is opened to the vacuum port and the contrast port;

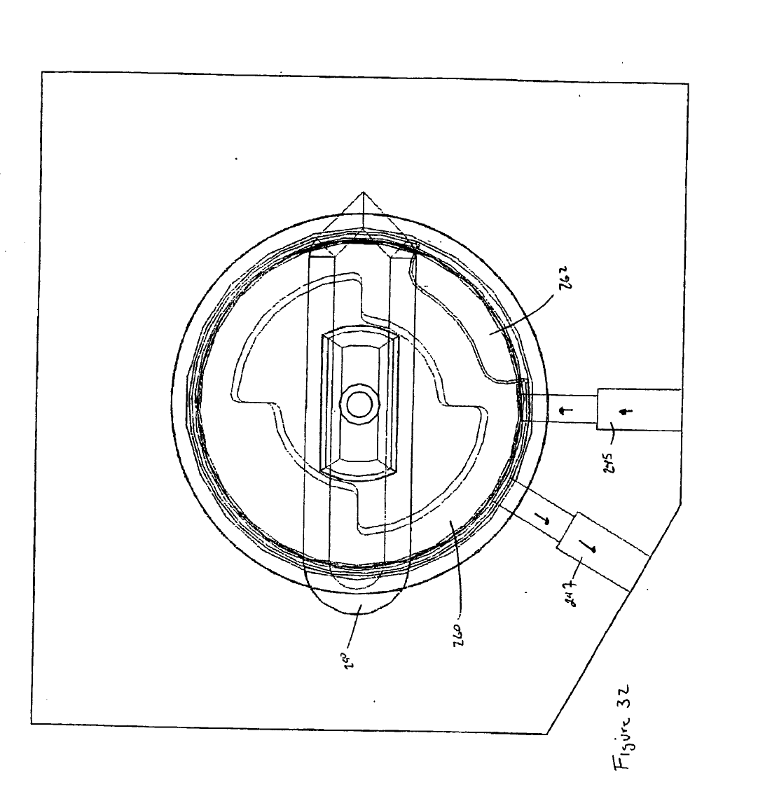

FIG. 32. is a top view of the alternative embodiment of the inflation knob in accordance with the present invention, wherein the selector valve has been moved to seal the vacuum port and the contrast port;

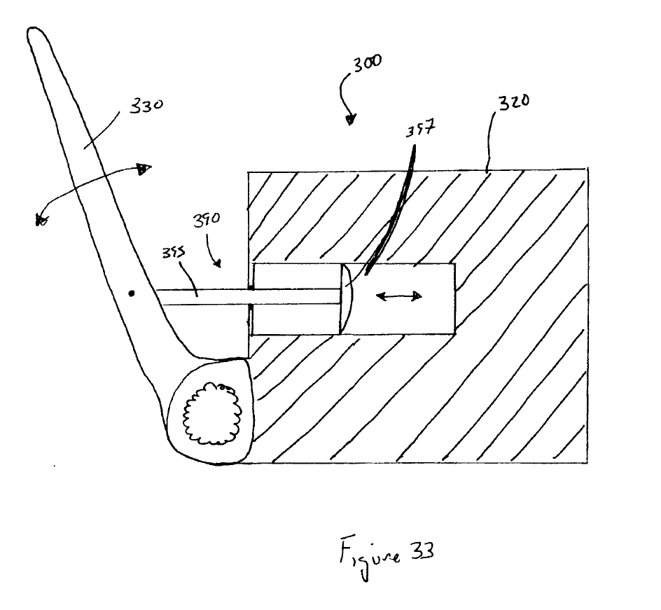

FIG. 33. is a cross-sectional view of yet another alternative embodiment of the inflation device in accordance with the present invention;

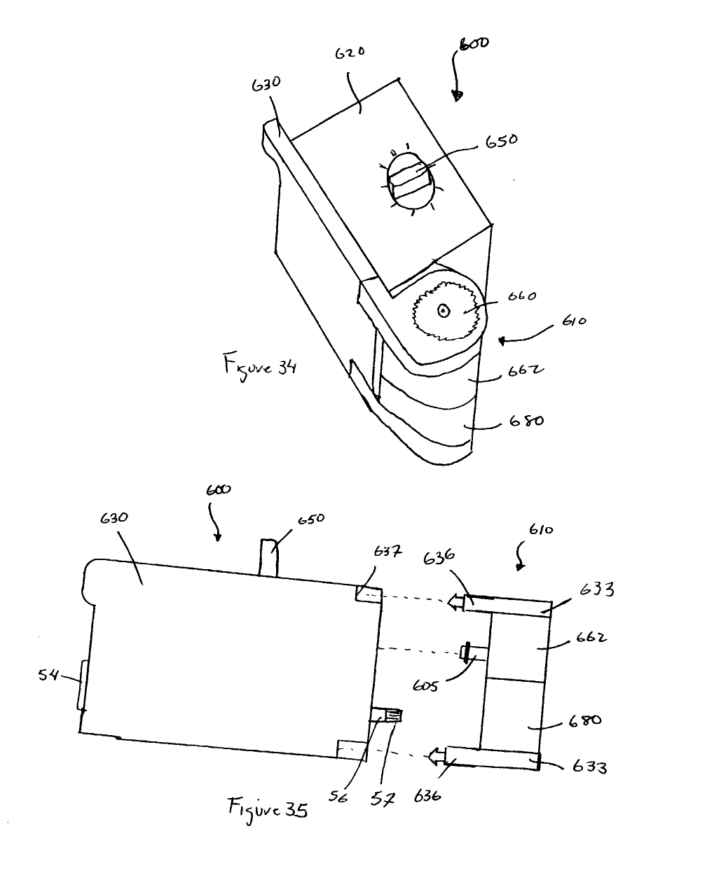

FIG. 34. is an expanded perspective view of an alternative embodiment of the inflation device in accordance with the present invention; and

FIG. 35. is a cross-sectional side view of the alternative embodiment of the inflation device illustrated in FIG. 34.

### DETAILED DESCRIPTION OF A PREFERRED EMBODIMENT

In accordance with the present invention, there is provided an inflation device for use with expandable medical devices such as those disclosed in co-pending U.S. patent application Ser. No. 09/822,823 filed on Jun. 15, 2001 entitled “Balloon Occlusion Device Having a Proximal Valve”, the entirety of which is herein incorporated by reference. The inflation device includes a main body, a locking knob, a locking knob rod, a seal lever, first and second seal knobs, a fluid chamber, an inflation control knob, a fluid inlet, a vacuum inlet, a selector knob, and a shuttle for opening and closing a valve assembly of a medical device.

Referring now to FIG. 1, there is shown a perspective view of the inflation device 10 in accordance with the present invention. As shown in FIG. 1, the seal lever 30 is shown in a closed and locked position. Referring now to FIG. 2, there is shown a perspective view of the inflation device 10 wherein the seal lever 30 is in an open unlocked position, wherein the inflation device 10 is capable of receiving a medical device (not shown) within the aperture 70 for inflation of an inflatable member disposed upon the medical device. As shown in FIGS. 1 and 2, the inflation device 10 further includes a main body 20 having a fluid chamber 53, a vacuum port 47, a fluid inlet 45, a chamber 64 for receiving a locking knob, and a projection 62 extending from the distal end portion 22. Each element of the inflation device 10 will be described in greater detail below with reference to the corresponding figures.

Referring now to FIG. 3, there is shown a side view of the inflation device 10 in accordance with the present invention, wherein FIG. 3 illustrates the inflation device 10 as assembled for use. Referring now to FIG. 4, there is shown a top view of the inflation device 10 in accordance with the present invention illustrating the various parts as assembled upon the main body 20 of the inflation device 10.

Referring now to FIG. 5, there is shown a side view of the main body 20 in accordance with the present invention. The main body 20 includes a proximal end portion 21, and a distal end portion 22. Wherein as shown in FIG. 5, the selector knob 40, inflation knob 50, and the first locking knob 60 have been disposed within the main body 20. The main body 20, further includes a plurality of conduits 57, as shown in FIGS. 7 and 8, disposed therethrough, wherein the conduits 57 are in fluid communication with the fluid inlet 45, vacuum inlet 47, fluid chamber 53, and the distal end 65 of the extension 67 protruding from the projection 62. The function of the conduits 57 will be described in greater detail below with reference to the use of the inflation device 10.

Referring now to the projection 62 extending from the distal end portion 22 of the main body 20, as shown in FIGS. 5 and 6. The projection includes an aperture 61 (not shown) for receiving a first seal knob 60, wherein the first seal knob 60 may be threadably disposed within the chamber 61. The first seal knob 60 engages a seal 75 disposed within a chamber 61 of the projection 62, wherein, as the first seal knob 60 is advanced into the chamber 61 the seal 75 is compressed. The projection 62 further includes an extension member 67, wherein the extension member 67 includes a seal 66 disposed radially thereabout as shown.

Referring now to FIG. 7, there is shown a cross-sectional side view of the main body 20 in accordance with the present invention. As shown in FIGS. 7 and 8, the fluid chamber 53 is in fluid communication with end of the extension member 67, and the vacuum port 47 (not shown) through the conduits 57 disposed within the main body 20. Additionally, the fluid inlet port 45 is in fluid communication with the end of the extension member 67, therein coupling the vacuum port 47 to the fluid inlet port 45 and to the fluid chamber 53.

In an alternative embodiment, the fluid inlet port may not be necessary; the inflation device may be pre-filled with contrast solution where all air within the fluid reservoir and associated conduits has been removed. In this embodiment, the vacuum port may also be eliminated because it would not be necessary to draw a vacuum in order to eliminate air within the fluid chamber or conduits because this would have been accomplished during the manufacture of the device. In yet another embodiment, it is contemplated that the fluid inlet may be replaced by an assembly that is designed to accept a pre-filled fluid cartridge, wherein the assembly includes a means for piercing a seal on the pre-filled cartridge therein emptying the contents of the cartridge into the inflation device. Additionally, the use of the terms “fluid” or “contrast” shall be understood to define any fluids that my be utilized to inflate the balloon disposed on the medical device. For example, fluids utilized to inflate balloons during surgical procedures include carbon dioxide, saline and similar fluids.

The main body 20 may be constructed of biocompatible materials such as titanium, stainless steel or plastics. In a preferred embodiment the main body is constructed of a biocompatible plastic such as polycarbonate. In a preferred embodiment the main body is manufactured as a unitary body as shown in FIGS. 1, 2, and 3. It is further contemplated that the main body may be constructed of a plurality of pieces that may be assembled utilizing a biocompatible adhesive, sonic welding, or similar procedures. Still further, in a preferred embodiment the main body 20 may be constructed wherein the main body is clear or opaque, therein allowing an operator to visually view the functionality of the inflation device 10 as well as visually determine if any air bubbles remain in the conduits 57 or fluid chamber 53. Additionally, the user may visually determine if the medical device is leaking after inflation because the fluid level in the fluid chamber 53 may decline without movement of the inflation knob 50.

Referring now to FIG. 8, there is shown a cross-sectional side view of the projection 62 of the main body 20 in accordance with the present invention. As shown in FIG. 8, the projection 62 includes an aperture 70 and a chamber 61 disposed therein. The chamber 61 is adapted to receive a first seal knob 60 as described above, wherein the first seal knob 60 contains an aperture 70 extending from the proximal end portion to the distal end portion. The chamber 61 is further adapted to receive a seal 75 as shown. The seal 75 further includes an aperture 70 disposed therethrough, wherein the apertures of the seal, seal knob and projection are in axial alignment. The chamber 61 is further adapted to engage the first seal knob 60. For example, the first seal knob 60 and chamber 61 may be threaded respectively, wherein the first seal knob 60 may be advanced within the chamber 61 by applying a radial force to the proximal end portion of the first seal knob 60. It is contemplated that other methods may be utilized to engage the first seal knob within the chamber, therefore the example above should not be considered limiting in any manner and should be considered exemplary. In the event that a threaded connection is utilized to advance or withdraw the first seal knob 60 within the chamber 61 of the projection 62, an appropriate thread must be utilized. That is, the thread pitch chosen must be sufficient to advance or retract the first seal knob 60 a sufficient amount during use as will be described in greater detail below.

The first seal knob 60 may be constructed of a biocompatible material such as those listed above. In a preferred embodiment, the first seal knob 60 is constructed of biocompatible plastic such as delrin, polycarbonate, nylon or similar biocompatible plastics, which may be sterilizable. Additionally, the first seal knob 60 may be manufactured using known techniques such as injection molding or machining.

As shown in FIG. 8, the seal 75 is disposed within the chamber 61 of the projection 62, wherein the seal 75 is sized accordingly to contact the walls of the chamber 61 when disposed therein. The seal 75 may be constructed of biocompatible materials such as silicone, urethane, delrin, rubber, latex, pebax, kraton, alcryn, and other similar materials that are conventionally utilized to construct seals in medical devices. The seal 75 is constructed having a diamond shaped cross-sectional profile. The diamond cross-sectional profile of the seal 75 is important, in that when the first seal knob 60 compresses the seal, the aperture 70 disposed through the seal compresses and becomes smaller thus gripping anything passed through the aperture formed in the seal.

As shown in FIG. 8 and described above, the projection 62 further includes an extension 67, wherein the aperture 70 is axially disposed through the extension 67 as shown. Additionally, the extension 67 further includes a gasket seal 66 disposed radially thereabout and adjacent a distal end portion. The projection 62 further includes a first fluid line 57 and a second fluid line 57′ disposed adjacent the aperture 70. The first and second fluid lines are respectively coupled to the fluid inlet, fluid chamber and vacuum inlet as will be described in greater detail below with reference to the methods of use of the inflation device 10.

Referring now to FIGS. 9-11, there is shown the shuttle assembly 80 in accordance with the present invention. The shuttle assembly 80 includes a shuttle body 82, shuttle 87 and second seal knob 85. The shuttle 87 further includes a first chamber 88 having an aperture 89 disposed therein, and a second chamber 88′ wherein the second chamber 88′ is adapted to receive a seal 75 as described above. The shuttle 87 is slidably disposed within the shuttle body 82. As shown in FIGS. 9-11, the shuttle 87 may include at least one groove 83 in order to align the shuttle properly within the shuttle body 82. In addition to the groove, the shuttle 87 may have a geometric shape such that the shuttle may not rotate within the shuttle body 82 when the shuttle is slidably disposed therein. The shuttle body may further contain chambers 81, wherein the chambers 81 may be threaded to receive at least one screw or bolt that may be utilized to retain the shuttle assembly when the shuttle assembly is assembled with the main body 20. Alternatively, it is contemplated that other methods may be utilized to secure the shuttle assembly 80 to the main body 20.

As shown in FIG. 10, the first chamber 88 disposed within the shuttle is adapted to receive the extension 67 of the projection 62. The gasket seal 66 radially disposed about the extension 67 forms a fluid tight seal between the extension 67 and the chamber 88, therein forming a valve chamber 90. It shall be understood that the projection, shuttle assembly and their respective related components maybe referred to additionally herewith as the inflation chamber.

Referring now to FIG. 12, there is shown a side view of the shuttle 87 in accordance with the present invention. As shown in FIG. 12, the shuttle 87 further includes a plurality of gear teeth 86 disposed within the sidewall of the shuttle 87. Additionally, as shown, the distal end portion of the shuttle is adapted to receive a second seal knob 85. The second locking knob 85 may be threadably engaged within the distal end portion of the shuttle 87. In a preferred embodiment the first and second seal knobs are threadably engaged within the corresponding structures. In addition to being threadably engaged within their corresponding structures, each seal knob and corresponding structure is threaded in opposite directions. That is in a preferred embodiment, the first seal knob and corresponding structure includes a right hand thread, which, when turned in a clockwise direction, the seal knob is advanced into the projection. In a preferred embodiment the second seal knob and shuttle have left hand threads, which, when turned in a counter clockwise direction the second seal knob is advanced into the shuttle. Thus, in a preferred embodiment, the seal knobs must have opposite direction advancing mechanisms. The purpose of the oppositely threaded seal knobs will be described in greater detail with reference to the methods of use of the inflation device.

Referring now to FIG. 11, there is shown a cross-sectional view of the shuttle assembly 80 according to the present invention. As shown in FIG. 11 and described above, the first chamber 88 of the shuttle 87 is adapted to receive the extension 67 and form a fluid tight seal therein. Additionally, the shuttle includes a second chamber 88′, wherein a seal 75 such as the one described above is disposed therein. The seal is compressed by the advancement of the second seal knob within the second chamber 88′ of the shuttle 87. The compression of the seal 75 by the second seal knob therein causes the aperture disposed through the seal to compress and grip anything disposed through the aperture. The shuttle body 82 may further include an aperture (not shown) disposed adjacent the gear teeth 86 therein exposing the gear teeth to a (seal) locking knob projecting from an aperture 64 disposed within the main body 20 of the inflation device as will be described in greater detail below.

The shuttle body 82 may be constructed of biocompatible materials such as those described above. In a preferred embodiment the shuttle body and second seal knob are constructed of biocompatible plastics such as polycarbonate or delrin, or polyvinyl chloride. The shuttle is preferably constructed of a biocompatible material such as titanium, stainless steel, injection-molded nylon, or similar plastics having good mechanical properties, which are, enable to withstand the torque of the locking knob.

Referring now to FIG. 13 there is shown the locking knob 55 in accordance with the present invention. As shown in FIG. 13 the locking knob includes an elongated shaft 56 extending from a knob portion 54. The distal end portion 57 of the elongated shaft 56 is adapted to engage the gear teeth 86 disposed upon the outer surface of the shuttle 87. The knob portion 54 of the locking knob includes means for locking the seal lever 30 of the inflation device 10 when the seal lever 30 is in a closed position.

The locking knob is rotatably disposed within the aperture 64 of the main body 20 of the inflation device 10. The distal end portion 57 extends beyond the distal end portion 21 of the main body 20, wherein the knob portion 54 of the locking knob is received within a relief formed within the proximal end portion of the main body 20. Additionally, the locking knob 55 may further include a locking device (not shown). Wherein the locking device engages a locking surface of the seal lever 30, wherein when the locking device engages the locking surface the seal lever cannot be accidentally opened. This safety feature is important in that it prevents the accidental opening of the seal lever when the valve assembly of the medical device is open. For example, if the locking device was not present and the seal lever was accidentally opened during use when the valve assembly of the medical device is open, the fluid within the inflation device would be expelled through the aperture 70 instead of being utilized to inflate the balloon. Therefore, the balloon may not be inflated properly, or may become deflated during use.

Referring now to FIG. 14, there is shown the inflation knob 50 in accordance with the present invention. The inflation knob 50 includes a proximal end portion, a distal end portion and a plurality of threads 51 disposed therebetween. As shown in FIGS. 1, 2, and 7 the inflation knob 50 is disposed within the fluid chamber 53 of the main body 20. As shown in FIG. 7, the inflation chamber 53 further includes a first seal 54 and a second seal 55 disposed adjacent to a plurality of threads. The seals engage the distal end portion of the inflation knob 50 and provide a fluid tight seal between the fluid chamber 53 and the atmosphere. Additionally, the distal end portion of the inflation knob may be modified to increase or decrease the amount of fluid, which is held within, or displace from the fluid chamber. For example, if it is desired to increase the volume of fluid in the fluid chamber the distal end portion of the inflation knob would be made shorter, the converse is true if it were desirable to decrease the volume of fluid in the fluid chamber. Still further, by being able to adjust the volume of the chamber by varying the distal end portion of the inflation knob 50, the inflation device may be custom configured to various inflatable medical devices. Still further, it is contemplated that the inflation knob may be disposed within the fluid reservoir using other connection means. For example, the inflation knob may be slidaby disposed within the main body and in communication with the fluid reservoir.

Referring now to FIGS. 15 and 16 there is shown the seal lever 30 in accordance with the present invention. The seal lever includes a proximal end portion 31 a distal end portion 37. The distal end portion 37 further includes at least one projection 35 extending therefrom, wherein the projection 35 is adapted to be gripable for opening and closing the seal lever 30. The seal lever 30 further includes a groove (not shown) disposed adjacent the distal end portion, wherein the groove is adapted to receive a locking device disposed upon the locking knob 55, the function of which was described above.

Disposed at the proximal end portion 31 of the seal lever 30 are at least two apertures 32 and 34. The apertures 32 and 34 are adapted to receive the first and second seal knobs 60 and 85 respectively. As shown in FIG. 15, the apertures may be formed having a pattern such as a gear, wherein the outer diameter of the seal knobs are formed having a corresponding gear pattern such that when the seal lever 30 is assembled with the elements described above the form the inflation device 10, the radial motion of opening the seal lever is translated into linear motion of the seal knobs. It shall be understood that the gear pattern shown and described should be considered merely exemplary and should not be considered limiting in any manner. For example, other geometric shapes such as square, octagonal, pentagonal, etc. may be utilized.

The seal lever may be manufactured of biocompatible materials such as those listed above. In a preferred embodiment the locking cover is manufactured of a biocompatible plastic such as polycarbonate, polyvinyl chloride, delrin, or similar plastics, which are capable of sterilization. The seal lever may be manufactured using conventional manufacturing methods such as machining or injection molding.

Referring now to FIGS. 17-18, there is illustrated the inflation device 10 in use in accordance with the present invention. As previously described, the inflation device includes a main body, a seal lever, a plurality of seal knobs operatively coupled to the seal lever, an inflation control knob, a fluid inlet, a vacuum inlet.

The inflation device 10 prepared for use by assembling the various parts described above to form a functional unit. The inflation device is assembled by first attaching the shuttle assembly 80 to the main body 20 through the use of a fastening device (not shown). Examples of an appropriate fastening device may include screws, press-fit clips, one-way clips, adhesives, sonic welding, or similar methods or devices, which may be used to join two or more pieces together. Prior to assembling the shuttle assembly 80 with the main body 20, the shuttle 87 is disposed within the shuttle chamber formed within the shuttle body 82. Still further, prior to assembling the shuttle assembly 80 onto the main body 20, a seal 66 is disposed about the extension 67 of the projection 62. After assembling the shuttle assembly with the main body 20 of the inflation device, a valve chamber 90 is formed between the extension 67 and the first chamber 88 of the shuttle 87.

The locking knob 55 may then be inserted into a chamber 64 formed in the main body 20 of the inflation device 10. The gear teeth 57 disposed upon the distal end portion of the locking knob 55 couple with the gear teeth 86 on the shuttle 87. Wherein rotational motion of the locking knob will cause the shuttle to be linearly translated within the shuttle body 82.

The proximal end portion 31 of the seal lever 30 is then disposed about the first and second seal knobs, wherein the gear teeth within the apertures of the seal lever align with the gear teeth of the seal knobs.

Referring now to FIG. 17A there is shown the inflation device 10 in accordance with the present invention being prepared for use. As shown in FIG. 17A, a proximal end P of an inflatable medical device 99 is disposed within the inflation chamber of the inflatable device 10. Prior to insertion of the proximal end, the seal lever 30 is translated into an unlocked open position as shown. By placing the seal lever into the open position as shown, both the first and second seal knobs are advanced outwardly therein releasing pressure on the seals within the inflation chamber, therein allowing a medical device to be disposed within the inflation chamber.

Referring now to FIG. 17B, there is shown the inflation device 10 in accordance with the present invention, wherein a syringe filled with a contrast solution is prepared and coupled to the fluid inlet 45. The fluid inlet may comprise a luer fitting or other standard fitting that may be coupled with a conventionally available syringe. A second syringe is coupled to the vacuum inlet 47, wherein the vacuum inlet may comprise a luer fitting or similar fitting which may be coupled to a conventional syringe. Prior to drawing a vacuum, the seal lever is closed and locked as shown in FIG. 18, closing the seal lever advances the first and second seal knobs, therein compressing the seals about the diameter of the medical device therein forming a fluid tight chamber. In addition to forming a fluid tight chamber, the rotation of the locking knob 55 advanced the shuttle assembly, therein opening a low profile valve disposed on the proximal end of the inflatable medical device.

After closing and locking the seal lever as described above a vacuum is drawn by pulling back on the plunger of the vacuum syringe, the selector knob 40 is then turned to open the contrast valve wherein contrast flows from the contrast syringe through the plurality of conduits 57, into the inflation chamber and fluid reservoir and then into the vacuum syringe. At this time the inflation knob is then turned to start position and stops the flow of contrast into the inflation device. The contrast valve is then closed wherein the vacuum syringe and the contrast syringe may then be detached from the inflation device.

The inflatable medical device 99 may then be removed from the inflation chamber by twisting the locking knob 55 counter-clockwise, wherein the low profile valve assembly of the inflatable medical device is closed. The seal lever 30 may then be rotated to release the proximal end of the inflatable medical device from the inflation chamber. At this time, the inflation device is considered to be primed and ready for use.

After the inflatable medical device 99 is placed within the patient at a desired location using known conventional techniques, the proximal end P of the inflatable device may then be re-inserted into the inflation chamber, the seal lever rotated to engage the seals and the locking knob turned clockwise to open the low profile valve assembly disposed on the proximal end of the inflatable medical device. The inflation knob may then be rotated to inflate the balloon disposed on the inflatable medical device to a known diameter. The inflatable balloon disposed on the inflatable medical device is inflated to a known diameter through the use of a known volume of fluid. Unlike conventional inflatable medical devices that are inflated to a desired diameter utilizing an unknown volume of fluid, the inflation device 10 in accordance with the present invention is configured to provide consistent inflation diameter of the balloon through the use of a known volume of fluid.

The inflatable medical device 99 shown and described above having a low profile valve disposed upon the proximal end portion, may be the device shown and described in U.S. patent application Ser. No. 09/822,823 filed one Jun. 15, 2001 entitled “Balloon Occlusion Device Having a Proximal Valve” the entirety of which is incorporated by reference, is inserted into the valve chamber 90 of the proximal end portion and advanced until the proximal end of the medical device 100 contacts the second seal knob 85.

Referring now to FIG. 19 there is shown an exemplary embodiment of an alternative inflation device in accordance with the present invention. As shown in FIG. 19 the inflation device 100 comprises a main body, a seal lever, a seal knob, an inflation port, a contrast inlet, a vacuum port, and an inflation knob.

In accordance with the exemplary alternative embodiment of the inflation device 100 in accordance with the present invention, similar reference numbers are utilized to denote similar elements as described above with regard to the inflation device 10.

As shown FIG. 19, there is shown a perspective view of the inflation device 100 in accordance within an alternative embodiment according to the present invention. As shown in FIG. 19, the seal lever 130 is shown in a closed and locked position. Referring now to FIG. 20, there is shown a side view of one side of the inflation device 100. As shown in FIG. 20, the inflation device includes a contrast inlet and a vacuum port, each of which will be described in detail below.

Referring now to FIG. 21, there is shown a cross-sectional view of the inflation device 100 in accordance with the present invention. As shown in FIG. 21, the inflation device 100 further includes a projection 162 and a shuttle assembly 180, each of which define an inflation chamber, wherein the inflation chamber is configured to receive the proximal end of an inflatable medical device. The inflation device further includes a fluid chamber 153, wherein the fluid chamber 153 is in fluid communication with the inflation chamber, the contrast inlet and the vacuum port through a plurality of conduits 157. Referring now to the projection 162 as shown in FIG. 21, the projection 162 further includes an extension member 167. The extension member 167 includes a seal 166 disposed adjacent to a distal end thereof, wherein the extension member and the seal is configured to receive and retain the shuttle assembly as shown in FIGS. 19 and 20. The extension member 167 further includes a plurality of conduits 157 and 157′ formed therein, wherein the conduits are in fluid communication with the fluid chamber 153. The projection 162 is further configured to threadably receive a first seal knob 160. The proximal end of the first seal knob 160 is configured to be engaged by a portion of the seal lever 130 as shown in FIG. 19. The first seal knob 160 further includes an aperture 170 disposed therethrough. The aperture 170 forms a portion of the inflation chamber and is configured to receive a proximal end of an inflatable medical device. The projection 162 further includes a seal 175 disposed at the proximal end of the first seal knob 160. The seal 175 includes an aperture formed therethrough, wherein the aperture is sized to accept the proximal end of an inflatable medical device therethrough, wherein the seal when compressed by the first seal knob both retains and fluidly seals about the medical device passed therethrough.

Still further, as shown in FIG. 21, the inflation device 100 includes a locking knob 155, wherein the locking knob has a proximal end and a distal end. A plurality of gear teeth 144 being formed at the proximal end and a knob 154 being formed at the distal end. The locking knob 155 further includes retaining means 146, wherein the retaining means may comprise a raised portion formed along a portion of the shaft 156 of the locking knob 155. As shown in FIG. 21, the retaining means is received within a corresponding groove 101 formed in the housing of the inflation device 100. The retaining means 146 is configured to detachably retain the locking knob 155 within the main body 120 of the inflation device 100, while further allowing the disassembly of the locking knob from the inflation device 100. The retaining means 146 ensures that during use, the user cannot accidentally pull back on the locking knob therein disassembling the inflation device 100. Referring now to the distal end of the locking knob wherein the knob 154 is disposed thereon. As shown in FIG. 21, the knob 154 further includes means for limiting the rotation of the locking knob 155. The limiting means includes a groove 143 formed in the knob portion 154 and a projection 123 formed within the main housing 120. In use, the groove 143 interfaces with the projection 123, thus limiting the rotational travel of the locking knob 155.

Referring now to FIG. 22 there is shown a cross-sectional view of the shuttle assembly 180 in accordance with the present invention. As shown in FIG. 22, the shuttle assembly comprises a shuttle body 182 and a shuttle 187. The shuttle body 182 further includes a first chamber 188 and a second chamber 188′. The first chamber 188 is configured to receive the extension 167 of the projection 162. The seal 166 disposed on the end of the extension 167 is slidably received within the chamber 188 therein forming a fluid tight seal within the chamber. As shown in FIG. 22, the second chamber 188′ is configured to receive a second seal knob 191, a seal 198, and a seal retaining member 197. The first and second chambers are coupled by an aperture 189, wherein the aperture is sized and configured to receive the proximal end of a medical device therethrough. In addition, the seal 198 and the seal-retaining member 197 each include an aperture formed therethrough.

As shown in FIG. 22, a first side of the seal 198 is formed having a concave surface, wherein the second side of the seal is formed having a substantially flat surface, wherein the flat surface of the seal is disposed against the seal-retaining member. The seal 198 may be formed of materials such as silicone, kraton, pebax, and other materials that are suitable for seals. The seal-retaining member 197 is formed of a rigid or substantially rigid material such as plastic, aluminum, stainless steel or other suitable materials.

The second seal knob 191 is configured to be threadably received within the second chamber 188′. The threads disposed on the second seal knob 191 are opposite those formed on the first seal knob 160. For example the first seal knob 160 may have right hand threads, thus the second knob would be formed having left hand threads, thus when the seal lever 130 is moved between an opened position and a closed position, each seal knob is advanced or retraced from the inflation device 100.

As shown in FIG. 22, the proximal end of the second seal knob 191 is configured to receive the seal lever 130 as shown in FIG. 19. The distal end of the second seal knob 191 includes a collet 195 formed therein. The collet 195 and the second seal knob 191 may be integrally formed or alternatively the collet 195 and the second seal knob 191 may be formed as separate pieces which are then joined utilizing know methods such as friction fit, bonding, welding, melting, sonic welding, or other similar processes.

Referring now to FIGS. 23, there is shown a perspective view of the second seal knob 190 in accordance with the present invention. As shown, the collet 195 includes a plurality of grooves 197 formed therein and an aperture 196. As the second locking member 190 is threadably advanced into the second chamber 188′ of the shuttle 187, the plurality of grooves 197 formed in the collet are compressed against the seal retaining member 197, thus closing and or reducing the diameter of the aperture 196 and compressing the seal 198, therein closing and/or reducing the diameter of the aperture disposed through the seal. It shall be understood that the projection, shuttle assembly and their respective related components maybe referred to additionally hereafter as the inflation chamber.

Referring now to FIGS. 24, there is shown a cross-sectional view of the inflation chamber of the inflatable medical device 100 in accordance with the present invention. As shown in FIG. 24, a proximal end of an inflatable medical device 99 has been disposed within the inflation chamber. As shown, the proximal end of the medical device 99 passes through the aperture 170 formed in the first seal knob 160, through seal 175, through seal 198 and seal retaining member 197, and into the aperture 196 formed in the second seal knob 190. As shown in FIG. 24 the first and second seal knobs have been advanced by rotating the seal lever to a closed position, therein compressing the seals. The seal 175 forms a fluid tight seal about the medical device and further retains the shaft of the medical device within the inflation chamber. The seal 198 is compressed by the second seal knob 190, wherein the second seal forms a fluid tight seal about the valve portion of the medical device. As shown in FIG. 25, the distal end of the medical device is retained within the collet 195 of the second seal knob, thus, when the locking knob 155 is activated the shuttle assembly is translated, therein opening the low profile valve of the medical device as shown. As shown in FIG. 24, the shuttle assembly is configured to translate with respect to the inflation chamber. By configuring the shuttle device to translate, a low profile valve assembly disposed on the proximal end of the inflatable medical device may be moved from a closed position to an open position, therein exposing the inflation lumen of the inflatable medical device. As shown in FIG. 24, the collet of the second seal knob is configured to grip and retain a portion of the valve assembly.

Referring now to FIG. 25, there is shown the inflation device 100 prior to use. As shown in FIG. 26, a syringe filled with a contrast solution is coupled to the fluid inlet 145, wherein a valve assembly 300 is fitted between the syringe and the inflation device. The valve assembly 300 allows a user to open and close the fluid path between the contrast syringe and the fluid inlet. A vacuum syringe is shown coupled to the vacuum port 147, and a medical device 99 is shown disposed within the inflation chamber of the inflation device 100 in accordance with the present invention.

In use, the user prepares the inflation device according to the following procedure. The inflation device 100 is removed from the packaging material; if the seal lever is disposed in the closed position, the knob portion of the locking knob is rotated counter clockwise unlocking the seal lever 130. The seal lever is then rotated away from the main body 120 of the inflation device 100, therein uncompressing the seals disposed within the inflation chamber. Alternatively, the inflation device may be shipped wherein the seal lever is already in the opened position. The proximal end of a medical device 99 is then placed within the inflation chamber. The seal lever 130 is then rotated to the position shown in FIG. 26, therein compressing the seals about the diameter of the medical device and compressing the collet about the proximal end of the medical device.

The user then rotates the knob portion of the locking knob 155 clockwise. The rotation of the locking knob engages a locking mechanism disposed on the knob portion and the seal lever 130. The gear teeth on the locking knob 155 engage a plurality of gear teeth on the shuttle body 182, therein the rotation of the gear teeth causes the shuttle body 182 to be displaced within the shuttle assembly, therein opening the sealing member of the valve assembly disposed on the proximal end of the medical device.

The inflation knob 150 is turned to the “open” position and the valve assembly 300 is turned to a closed position. The user then pulls back on and locks the plunger of the vacuum syringe therein developing a vacuum within the plurality of channels 157, 157′, the fluid reservoir 153, the inflation chamber and within the medical device. The user then opens the valve assembly 300 therein allowing contrast to flow from the contrast syringe into the plurality of channels, fluid reservoir, inflation chamber, medical device, and out into the vacuum syringe. During this process, the user will see air bubbles entering the vacuum syringe, as soon as the amount of air bubbles significantly changes or become no longer visible in the fluid entering the vacuum syringe the user then rotates the inflation knob to the “start” position and closes the contrast valve disposed on the contrast valve disposed on the contrast syringe. The vacuum syringe and the contrast syringe can then be removed from the inflation device.

The user may at this time inflate the balloon disposed on the inflatable medical device by rotating the inflation knob and visually inspecting the inflation of the balloon. After testing the inflation of the balloon, the user may then rotate the inflation knob to the “deflate” position or back to the start position, wherein the balloon disposed on the inflation device may then be deflated. After checking for proper inflation and deflation of the balloon, the inflation device is prepared and ready to be used. The medical device may then be removed from the inflation device, wherein the locking knob is rotated counterclockwise, therein causing the shuttle body to be translated therein closing the valve assembly disposed on the medical device. The rotation of the locking knob additionally releases the locking mechanism therein allowing the seal lever 130 to rotate. The seal lever 130 may then be rotated therein releasing pressure on the seals and collet; the medical device can then be removed from the inflation chamber of the inflation device.

The function of the inflation device may be better understood with reference to the functional flow diagram illustrated in FIG. 26, wherein the functional flow diagram illustrates the steps describe above for preparing the inflation device for use. Referring now to Box 400, the locking knob is rotated counter clockwise and the seal lever is rotated therein opening the inflation chamber. Referring now to Box 410 the proximal end of an inflatable medical device is inserted within the inflation chamber. Referring now to Box 420, the seal lever is rotated clockwise to a closed position, and the locking knob is rotated clockwise to lock the seal lever in place and open the low profile valve disposed on the proximal end of the inflatable medical device. Referring now to Box 430, a contrast syringe and a vacuum syringe are removably connected to the respective ports on the inflation device. Referring now to Box 440, the plunger of the vacuum syringe is pulled back, therein forming a vacuum within the lumen of the inflatable medical device, inflation chamber, fluid chamber and respective conduits interconnecting each of the above, the plunger is affixed to retain the vacuum within the syringe. Referring now to Box 450 the stopcock or valve disposed between the contrast syringe and the contrast inlet is rotated to allow contrast to flow from the contrast syringe through the inflation device and inflatable medical device. Referring now to Box 460, the inflation knob is rotated to a “start” position, wherein the contrast inlet and the vacuum outlet are fluidly sealed. Referring now to Box 470 the contrast syringe and stopcock (valve) and the vacuum syringe are removed from the respective ports disposed on the inflation device. Referring now to Box 480, the inflation knob is rotated from the “start” position to “3 mm” to inflate the balloon disposed on the distal end of the inflatable medical device to check for leaks and to prepare the balloon for use. Referring now to Box 490 the inflation knob if rotated from the “3 mm” mark back to “start” or “deflate”, wherein the balloon is deflated. Referring now to Box 500, the locking knob is rotated counter-clockwise to close the valve assembly on the inflatable medical device and unlock the seal lever, the seal lever is then rotated counter-clockwise to release the pressure on the seals in the inflation chamber, wherein the proximal end of the inflatable medical device may then be removed from the inflation chamber. Referring now to Circle 510, the inflation device is primed and ready for use.

The inflatable medical device 99 can then be placed into the patient's arterial system to a desired location utilizing known placement methods such as flouoscopy. When it is desirable to inflate the balloon disposed on the medical device, the proximal end of the medical device is inserted into the inflation chamber; the seal lever is rotated against the body of the inflation device and locked by rotating the locking knob. The inflation knob 150 can then be rotated to inflate the balloon to a desired diameter as indicated in FIG. 28 by the arrow labeled “I”. The balloon can be inflated and deflated by rotating the inflation knob clockwise or counterclockwise as desired by the user, as shown in FIGS. 26 and 28, the inflation device 100 may include markings on the top surface adjacent to the inflation knob, wherein the markings indicate known balloon diameters or known functions such as “start”, “deflate”, or “open”. The inflation device 100 will remain primed and ready for use, that is the seal lever and locking knob may be rotated between an opened and closed position multiple times while inserting and removing the proximal end of the medical device from the inflation chamber, so long as the user does not rotate the inflation knob to the “open” position after priming the device according to the procedure above.

Referring now to FIG. 28, there is shown a functional flow diagram illustrating the method of using the inflation device 100 for inflating a balloon disposed on an inflatable medical device. Referring now to Box 520, the proximal end of the inflatable medical device is disposed within the inflation chamber. Referring now to Box 530, the seal lever is rotated counter clockwise to engage the seals in inflation chamber, and the locking knob is rotated clockwise to lock the seal lever and open the valve assembly of the inflatable medical device. Referring now to Box 540, the inflation knob is then rotated from the “start” or “deflate” position to a marked balloon diameter location. Referring now to Diamond 550 it is then determined if the inflatable medical device is to be removed or retained within the patient. If the device is not to be retained, then proceed to Box 555 wherein the inflation knob is rotated to the “deflate” position, and the locking knob and seal lever are rotated counter-clockwise to release the proximal end of the inflatable medical device. After rotating the inflation knob to deflate, proceed to Box 580, wherein the locking knob and seal lever are opened to allow removal of the proximal end of the medical device from the inflation chamber, then proceed to Diamond 590 to determine if the device is to be reinflated/reinserted. Referring now to Diamond 560 it is determined whether or not the proximal end of the inflatable medical device is to be removed from the inflation chamber of the inflation device. If the proximal end is to be removed then proceed to Box 556, wherein the locking knob is rotated counterclockwise to close the valve assembly of the inflatable medical device and unlock the seal lever, then proceed to Box 580. At Box 580, the seal lever may then be rotated counterclockwise to release the proximal end of the inflatable medical device from the inflation chamber while retaining the balloon in an inflated state, when it is desired to deflate or increase the diameter of the balloon then proceed to Diamond 590 or Box 520. If it is determined in Diamonds 550 and 560, that the device is not to be removed from the patient or the proximal end is not to be removed from the inflation device, then proceed to Box 570. At Box 570, the size of the balloon may be adjusted by rotating the inflation knob clockwise or counterclockwise to increase or decrease the diameter of the balloon, after the balloon has been adjusted return to Diamond 550.

Referring now to FIGS. 29-32, there is shown an alternative embodiment of the inflation knob in accordance with the present invention. As shown in FIGS. 27-30, the inflation knob assembly 205 includes an inflation knob 250, a seal 260 and a seal-retaining member 252. The seal 260 further includes a recessed portion 262 and an aperture 263 formed therethrough. The seal 260 may be constructed of materials such as silicone, kraton or other similar materials suitable for forming seals. Alternatively, the seal 260 may be formed of a rigid or semi-rigid member including a seal disposed thereabout as will be apparent to one skilled in the art. As shown in FIG. 27, the inflation knob assembly 205 is to be disposed within a fluid chamber formed in the main body of the inflation device, a portion of which is shown in FIG. 27. The fluid chamber further includes a fluid inlet 245 and a vacuum outlet 247 as shown.

Referring now to FIG. 29 there is shown the inflation knob assembly 205 assembled within the fluid chamber of an inflation device. As shown, the recessed portion is aligned with the vacuum port 247, wherein a user may then pull back on the plunger of a syringe connected to the vacuum port thus developing a vacuum within the fluid chamber, inflation chamber, and the medical device.

Referring now to FIG. 30, after drawing a vacuum, the inflation knob is then rotated therein aligning the vacuum port 247 and the fluid inlet port 245, thus allowing fluid to flow from the fluid inlet port into the fluid chamber, inflation chamber and medical device and out through the vacuum port. After fluid has entered each of the above areas and flows out of the vacuum port, the user then rotates the inflation knob as shown in FIG. 30, therein sealing off the fluid inlet port and the vacuum port. The inflation device may then be utilized in accordance with that described above.

It has been determined that in use, the inflation knob assembly 205 may further require a fluid flow restrictor disposed within the fluid inlet. The fluid flow restrictor may comprise a hydrophilic material that swells when exposed to fluid. Alternatively, the fluid flow restrictor may comprise an insert having a smaller diameter disposed within the fluid flow path. The fluid flow restrictor being utilized to slow the flow of fluid through the fluid chamber, inflation chamber, medical device and out the vacuum port. The restrictor may be necessary because the user may not have enough time to rotate the inflation knob to the position shown in FIG. 32 to seal the fluid inlet and the vacuum port prior to draining the fluid from the syringe coupled to the fluid inlet and therein loosing the vacuum, and thus requiring re-priming of the system.

Referring now to FIG. 33 there is shown a cross-sectional view of yet another alternative embodiment of the inflation device in accordance with the present invention. As shown in FIG. 33 the inflation device 300 includes a main body 320, a locking cover 330, an inflation knob (not shown), and a fluid inlet (not shown). Additionally, as shown in FIG. 33, the inflation device 300 further includes means for generating a vacuum 390. As shown, the means for generating vacuum may comprise a plunger assembly 395. The plunger assembly 395 being coupled to the locking cover 330, wherein the motion of closing the locking cover advances a plunger 396 disposed within a chamber 397 formed within the main body of the inflation device 300. Although the plunger is illustrated as being directly coupled to the locking cover, it is contemplated that the plunger may be activated utilizing any known mechanical combination. For example, the plunger may be activated through the use of a series of gears, cables and pulleys, etc. Alternatively, the vacuum may be formed utilizing electromechanical means such as an electric motor coupled to a vacuum pump or similar means.

Referring now to FIG. 34, there is shown yet another alternative embodiment of the inflation device in accordance with the present invention. As shown in FIG. 34, the inflation device 600 includes a main body 620, a seal lever 630, an inflation knob 650, and a removable inflation chamber 610. The removable inflation chamber 610 defined by a projection 662 and shuttle assembly 680, wherein the projection and shuttle assemblies further include seal knobs 660 and 685 (not shown). The inflation device 600 functions in the same or similar manner to that as described above with regard to the inflation devices 100 and 10 and therefore will not be described in greater detail.

Referring now to FIG. 35 there is shown the inflation device 600, wherein the removable inflation chamber 610 is shown being detached from the main body 620 of the inflation device. As shown, the removable inflation chamber 610 includes projection 605 extending from the projection 662, wherein the projection 605 is configured to be received within an aperture formed in the main body 620. The projection 605 further includes a seal 606 disposed radially thereabout, wherein the seal 606 is configured to form a fluid tight seal between the inflation chamber and the fluid reservoir disposed within the main body 620. The removable inflation chamber 610 further includes seal lever sections 633 wherein the seal lever sections 633 a configured to receive the seal knobs 660 and 685 (not shown) in a similar manner to that shown in FIGS. 1 and 19. The seal lever sections 633 include projections 636, wherein the projections are configured to be received within grooves/apertures formed at the distal end 637 of the seal lever 630.

As shown in FIG. 35, the main body further includes seal knob 655, wherein a geared portion 567 of the seal knob extends beyond the distal end of the main body. The removable inflation chamber 610 includes an aperture configured to receive the geared portion of the seal know, wherein the geared portion is received within the shuttle assembly as described above. Although not shown, it is contemplated that the removable inflation chamber or the main body may include receiving means, wherein the receiving means are configured to detachably retain the two assemblies. For example, the receiving means may include assemblies such as a hook and a receiving slot, a protrusion and an aperture wherein the protrusion is frictionally received within the aperture.

As shown in FIGS. 34 and 35, the removable inflation chamber 610 is configured to allow the inflation device 600 to be manufactured in multiple components, some of which may be designed to be disposable (inflation chamber 610) and some of which may be designed to be reusable (main body 620, and related components). By forming the inflation chamber to be a removable replaceable assembly allows the inflation device to be configured to be utilized with other inflatable medical devices. For example, the aperture through which the proximal end of an inflatable medical device to be inflated may be made having differing diameters, wherein the proper diameter may be selected for use with the proper diameter inflatable medical device. Additionally, the removable section may reduce the cost of the device because only a portion of the device is disposed of after use. Still further, the shuttle assembly of the removable inflation chamber maybe configured to receive and actuate different valve assemblies disposed upon the inflatable medical device to be disposed within the inflation chamber.

Still further, it is contemplated that the removable inflation chamber 610 maybe configured to be coupled to an automatic inflation means (not shown). For example, the inflation chamber 610 maybe connected to a computer controlled console, wherein the console may be programmed or operated to inflate the balloon with a high degree of accuracy or inflate/deflate the balloon in combination with other surgical procedures.

Although the method of use of the inflation device has been described and shown in the above-referenced functional flow charts with regards to the inflation device 100. It shall be understood that the same or similar method may be utilized to use the inflation device 10 in accordance with the present invention. Wherein the functional steps of the inflation device 10 are similar to or the same as those described with regard to the inflation device 100 wherein one skilled in the art could easily understand the difference between the two devices. Further still, while the inflation device has been shown and described in use in preferred embodiments this shall not be considered limiting in any manner, it shall be understood that one skilled in the art may undertake modifications to the above reference device and methods of use without departing from the scope and nature of the present invention.

Although the present invention has been described according to preferred embodiments, this should not be considered limiting in any manner. For example it is contemplated that one skilled in the art may undertake modifications to the invention described herein without departing from the overall scope of the invention.

1. An inflation device, comprising:
a main body having a fluid reservoir;
a fluid inlet in fluid communication with said reservoir;
a vacuum port in fluid communication with said reservoir; and
an inflation chamber associated with said main body, said inflation chamber configured to receive an inflatable medical device therein, said inflation chamber including means for actuating a valve assembly of said inflatable medical device when said medical device is disposed within said inflation chamber.

2. The inflation device according to claim 1, wherein said means for actuating a valve assembly comprises a first seal knob, a second seal knob, wherein said first and second seal knobs compress at least one seal disposed within said inflation chamber to create a fluid tight seal between said fluid reservoir and said valve assembly.

3. The inflation device according to claim 2, wherein said means for actuating said valve assembly further includes a seal lever said seal lever coupled to said first and second seal knobs, wherein motion of said seal lever moves said first and second seal knobs to compress and uncompress said seals.

4. The inflation device according to claim 3, wherein said means for actuating said valve assembly further includes a shuttle assembly, the shuttle assembly slidably disposed within said inflation device and configured to threadably receive said second seal knob.

5. The inflation device according to claim 1, wherein one of said seal knobs further includes a collet.

6. The inflation device according to claim 4, wherein said inflation device further include a locking knob, said locking knob rotatably disposed within said housing and configured to retain said seal lever in a closed position, and wherein rotation of said locking knob opens and closes said valve assembly of said inflatable medical device.

7. The inflation device according to claim 1, wherein said inflation device further includes an inflation knob associated with said main body and in fluid communication with said fluid reservoir, said inflation knob configured to displace a known volume of fluid to inflate a balloon of a medical device when said medical device is disposed within said inflation chamber.

8. A device for inflating balloons disposed upon medical devices, the inflatable device comprising:
a main body having a fluid chamber;
an inflation knob associated with said fluid chamber;
a seal lever, rotatably associated within said main body;
a first seal knob disposed within the main body, wherein the seal knob is operatively coupled to said seal lever; and
a second seal knob disposed within a shuttle assembly coupled to the main body, wherein the second seal knob is operatively coupled to said seal lever, said first and second seal knobs define an inflation chamber, the inflation chamber in fluid communication with said fluid chamber,
said shuttle assembly coupled to one of said seal knobs, wherein the shuttle assembly is configured to open and close a valve assembly disposed upon a medical device when a valve assembly is inserted into said inflation chamber.

9. The inflation device according to claim 8, wherein the fluid chamber further includes at least one conduit in fluid communication with said inflation chamber.

10. The inflation device according to claim 8, wherein the first seal knob is threadably engaged within the main body, and the second seal knob is threadably engaged within the shuttle assembly.

11. The inflation device according to claim 10, wherein the threaded portion of the first seal knob is threaded opposite of said second seal knob.

12. The inflation device according to claim 8, wherein the locking knob and the seal lever further include means for locking the seal lever in a closed position.

13. The inflation device according to claim 12, wherein said locking knob further includes means for limiting rotational motion of the locking knob.

14. A method of inflating a balloon on a medical device, said method comprising:
(a) disposing an inflation port of a medical device within an inflation chamber of an inflation device;
(b) creating a vacuum within said inflation chamber of said inflation device and within an inflation lumen of said medical device, wherein said inflation lumen is in fluid communication with a balloon coupled to said medical device; and
(c) displacing a predetermined volume of inflation fluid to inflate said balloon to a known diameter after creating said vacuum.

15. The method according to claim 14, further including the step of providing a means for opening and closing a valve disposed upon said medical device, wherein said valve is coupled to said inflation lumen of said medical device.

16. The method according to claim 15, further including the step of closing said valve to retain said balloon at an inflated diameter.

17. The method according to claim 16, further including the step of removing said medical device from said inflation chamber while retaining said balloon in an inflated state.

18. The method according to claim 14, wherein the step of providing an inflation fluid includes connecting a fluid source to a fluid inlet on said inflation device to fill a reservoir within said inflation device, and wherein said fluid source is removed before the step of displacing fluid to inflate the balloon.

19. A method of inflating a balloon disposed upon a medical device, the medical device including a low profile valve and a balloon disposed upon a distal end portion, the method comprising:
providing an inflation device, the inflation device comprising a main body, a valve chamber, a device for opening an closing the valve chamber, and a device for opening the valve assembly of the medical device;
opening the valve chamber for insertion of a proximal end portion containing a low profile valve;
closing the valve chamber therein creating a fluid tight chamber about the valve assembly of the medical device;
opening the valve of the medical device;
providing an inflation knob coupled to a fluid reservoir, wherein a rotational force applied to the inflation knob causes fluid to enter the valve chamber and inflate the balloon;
closing the valve assembly of the medical device; and
releasing the proximal end portion of the medical device from the valve chamber.

20. An inflation device, comprising:
a main body having a fluid reservoir;
a fluid inlet in fluid communication with said reservoir;
a vacuum port in fluid communication with said reservoir; and
an inflation chamber associated with said main body, said inflation chamber configured to receive a valve assembly of an inflatable medical device therein, said inflation chamber includes an actuating device for acting upon said valve assembly when said medical device is disposed within said inflation chamber.

21. The inflation device according to claim 20, wherein said actuating device includes a first seal knob, a second seal knob, said first and second seal knobs configured to compress at least one seal disposed within said inflation chamber to create a fluid tight seal between said fluid reservoir and said valve assembly of said medical device when said medical device is disposed within said inflation chamber.

22. The inflation device according to claim 21, wherein said actuating device further includes a seal lever, said seal lever associated with said first and second seal knobs, wherein motion of said seal lever displaces said first and second seal knobs to compress and uncompress said seals.

23. The inflation device according to claim 22, wherein said actuating device further includes a shuttle assembly, the shuttle assembly slidably disposed within said inflation device and configured to threadably receive said second seal knob.

24. The inflation device according to claim 22, wherein one of said seal knobs further includes a collet.

25. The inflation device according to claim 24, wherein said inflation device further include a locking knob, said locking knob rotatably disposed within said housing and configured to retain said seal lever in a closed position, and wherein rotation of said locking knob opens and closes the valve assembly of the inflatable medical device.

26. The inflation device according to claim 20, wherein said inflation device further includes an inflation knob associated with said main body and in fluid communication with said fluid reservoir, said inflation knob configured to displace a known volume of fluid to inflate a balloon of a medical device when said medical device is disposed within said inflation chamber.
